(19) United States
(12) Patent Application Publication
(10) Pub. No.: US 2019/0180440 A1
(43) Pub. Date: Jun. 13, 2019

(54) DYNAMIC IMAGE PROCESSING DEVICE

(71) Applicant: Konica Minolta, Inc., Tokyo (JP)

(72) Inventors: Yuki MATSUMOTO, Osaka (JP); Hitoshi Futamura, Tokyo (JP)

(73) Assignee: Konica Minolta, Inc., Tokyo (JP)

(21) Appl. No.: 16/212,625

(22) Filed: Dec. 6, 2018

(30) Foreign Application Priority Data
Dec. 12, 2017 (JP) 2017-237282

(51) Int. Cl. G06T 7/00; A61B 6/00

(52) U.S. Cl. CPC G06T 7/0016; A61B 6/5211

(57) ABSTRACT

A dynamic image processing device includes: a hardware processor that sets a kind of a dynamic state to be diagnosed; extracts a frame image indicating the dynamic state of the kind set by the hardware processor from each of a plurality of dynamic images obtained by imaging a dynamic state of a living body with radiation; extracts a dynamic state dependent characteristic amount dependent on the dynamic state of the kind set by the hardware processor from the frame image extracted from each of the plurality of dynamic images; and standardizes the plurality of dynamic images based on the dynamic state dependent characteristic amount extracted by the hardware processor.

The entire disclosure of Japanese patent Application No. 2017-237282, filed on Dec. 12, 2017, is incorporated herein by reference in its entirety.

### BACKGROUND

### Technological Field

The present invention relates to a dynamic image processing device.

### Description of the Related art

Conventionally, a dynamic image obtained by imaging a dynamic state having periodicity of a subject with radiation is used for diagnosis. By using the dynamic image, it is possible to display and analyze the dynamic state of the subject which cannot be captured in a still image.

For example, JP 2012-192255 A discloses a technique in which a time-series change in a movement amount of a plurality of parts in a living body is detected based on a dynamic image, a time-series change in a movement amount of a predetermined part or a time-series change in a movement amount of another part is moved along a temporal axis direction by a predetermined amount, and a time-series change in a relative movement amount of the other part is obtained and displayed as having the time-series change in the movement amount of the predetermined part as a reference.

Meanwhile, at the time of diagnosis, there is a case where it is desired to perform comparative diagnostic reading on a specific kind of a dynamic state included in a plurality of dynamic images (for example, past and current dynamic images of the same patient) as a diagnosis target. For example, in general, a dynamic image of the chest includes any one of a plurality of kinds of dynamic states including a dynamic state accompanied with quiet breathing, a dynamic state accompanied with deep breathing, a dynamic state accompanied with heartbeat, and the like, and there is a case where it is desired to perform comparative diagnostic reading on any one of dynamic state as a diagnosis target. However, the technique described in JP 2012-192255 A has a problem in that, since the kind of the dynamic state to be diagnosed is not considered, a part of a time-series change in a dynamic state to be noted as a diagnosis target cannot be easily grasped. In addition, in the technique described in JP 2012-192255 A, although a temporal deviation occurred between the predetermined part and the other part in the dynamic image is considered, a deviation between the dynamic states of the plurality of dynamic images is not considered.

### SUMMARY

An object of the present invention is to easily perform comparative diagnostic reading on a dynamic state to be diagnosed included in a dynamic image.

To achieve the abovementioned object, according to an aspect of the present invention, a dynamic image processing device reflecting one aspect of the present invention comprises:
a hardware processor that
sets a kind of a dynamic state to be diagnosed;
extracts a frame image indicating the dynamic state of the kind set by the hardware processor from each of a plurality of dynamic images obtained by imaging a dynamic state of a living body with radiation;
extracts a dynamic state dependent characteristic amount dependent on the dynamic state of the kind set by the hardware processor from the frame image extracted from each of the plurality of dynamic images; and
standardizes the plurality of dynamic images based on the dynamic state dependent characteristic amount extracted by the hardware processor.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram of an overall configuration of a dynamic image processing system according to an embodiment of the present invention;

FIG. 2 is a flowchart illustrating imaging control processing executed by a controller of an i console in FIG. 1;

FIG. 3 is a flowchart illustrating comparison display processing A executed by a controller of a diagnostic console in FIG. 1 in a first embodiment;

FIG. 4 is a flowchart illustrating comparison display processing B executed by a controller of a diagnostic console in FIG. 1 in a second embodiment;

FIG. 5 is a flowchart illustrating comparison display processing C executed by a controller of a diagnostic console in FIG. 1 in a third embodiment; and

FIG. 6 is a flowchart illustrating comparison display processing D executed by a controller of a diagnostic console in FIG. 1 in a fourth embodiment.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

### [Configuration of Dynamic Image Processing System 100]

First, a configuration of a first embodiment will be described.

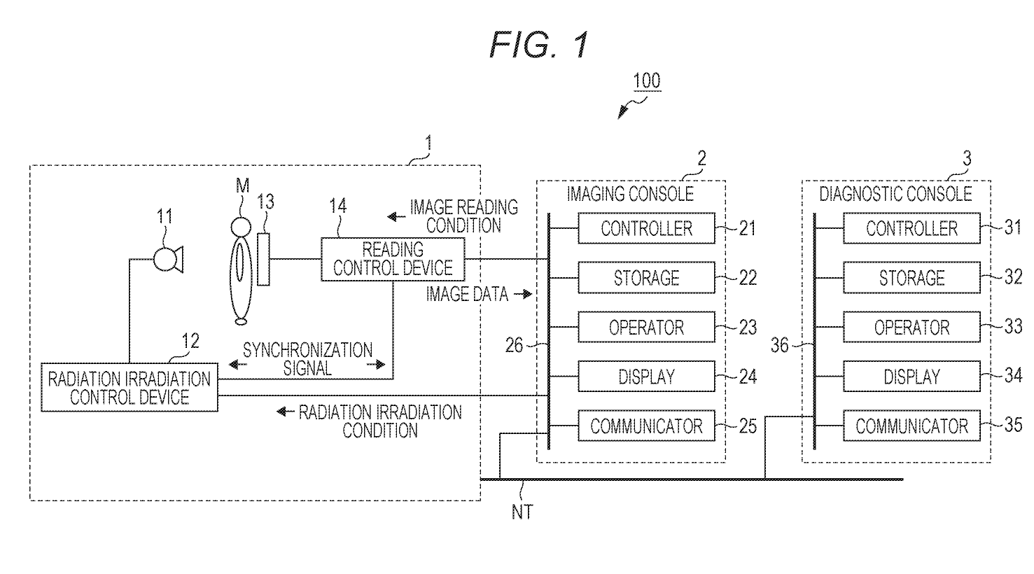

FIG. 1 illustrates an overall configuration of a dynamic image processing system 100 according to the first embodiment.

As illustrated in FIG. 1, the dynamic image processing system 100 includes an imaging device 1 and an imaging console 2 which are communicated with a communication cable and the like. The imaging console 2 is connected to a diagnostic console 3 via a communication network NT such as a local area network (LAN). The devices included in the dynamic image processing system 100 conform to the digital image and communications in medicine (DICOM) standard, and communication between the devices is performed in conformity with the DICOM.

### [Configuration of Imaging Device 1]

The imaging device 1 is an imager which images a periodic (cyclic) dynamic state, for example, a change in a form according to expansion and contraction of the lung accompanying with respiratory movement and beats of the heart. Dynamic state imaging is to obtain a plurality of images of a dynamic state of a subject by repeatedly irradiating the subject with pulsed radiation such as X-rays at predetermined time intervals (pulse irradiation) or continuously irradiating the subject with radiation with a low dose rate (continuous irradiation). A series of images obtained by the dynamic state imaging is referred to as a dynamic image. Furthermore, each of the plurality of images included in the dynamic image is referred to as a frame image. In the following embodiments, a case where a dynamic image of the chest is imaged by the pulse irradiation will be described as an example.

A radiation source 11 is arranged at a position facing a radiation detector 13 with a subject M therebetween and irradiates the subject M with radiation (X-rays) under control of a radiation irradiation control device 12.

The radiation irradiation control device 12 is connected to the imaging console 2 and performs radiation imaging by controlling the radiation source 11 based on a radiation irradiation condition input from the imaging console 2. The radiation irradiation conditions input from the imaging console 2 are, for example, a pulse rate, a pulse width, a pulse interval, the number of imaging flames per imaging processing, a value of an X-ray tube current, a value of an X-ray tube voltage, and a kind of an additional filter. The pulse rate is the number of times of radiation irradiation per second and coincides with a frame rate to be described later, The pulse width is a radiation irradiation time per radiation irradiation. The pulse interval is a time from start of the radiation irradiation to start of next radiation irradiation and coincides with a frame interval to be described later.

The radiation detector 13 includes a semiconductor image sensor such as an FPD. The FPD includes, for example, a glass substrate, and a plurality of detection elements (pixel), which detects radiation which has been emitted from the radiation source 11 and passed through at least the subject M according to the strength of the radiation, converts the detected radiation into electric signals, and accumulates the signals, is arranged in matrix at a predetermined position on the substrate. Each pixel includes a switcher such as a thin film transistor (TFT). There are an indirect conversion type FPD which converts X-rays into electric signals by a photoelectric conversion element via a scintillator and a direct conversion type FPD which directly converts X-rays into electric signals, and either one of the FPDs may be used.

The radiation detector 13 is provided to face the radiation source 11 with the subject M interposed therebetween.

A reading control device 14 is connected to the imaging console 2. The reading control device 14 controls the switcher of each pixel of the radiation detector 13 based on image reading conditions input from the imaging console 2, switches pixels from which the electric signals accumulated in the pixels are read, and reads the electric signals accumulated in the radiation detector 13. With this operation, the reading control device 14 obtains image data. This image data is a frame image. Then, the reading control device 14 outputs the obtained frame image to the imaging console 2. The image reading conditions are, for example, a frame rate, a frame interval, a pixel size, and an image size (matrix size). The frame rate is the number of frame images to be obtained per second and coincides with the pulse rate. The frame interval is a time from start of an operation for obtaining one frame image to start of an operation for obtaining the next frame image and coincides with the pulse interval.

Here, the radiation irradiation control device 12 and the reading control device 14 are mutually connected and synchronize the radiation irradiation operation and the image reading operation by exchanging synchronization signals.

### [Configuration of Imaging Console 2]

The imaging console 2 outputs the radiation irradiation conditions and the image reading conditions to the imaging device 1 to control the radiation imaging and the radiation image reading operation by the imaging device 1 and displays the dynamic image obtained by the imaging device 1 for confirmation whether the image is suitable for positioning confirmation, by a photographer such as a photographing engineer and diagnosis.

As illustrated in FIG. 1, the imaging console 2 includes a controller 21, a storage 22, an operator 23, a display 24, a communicator 25, and these parts are connected with a bus 26.

The controller 21 includes a central processing unit (CPU), a random access memory (RAM), and the like. The CPU of the controller 21 reads a system program and various processing programs stored in the storage 22 and develops the read program in the RAM in response to an operation of the operator 23, performs various processing including the imaging control processing to be described later according to the developed program, and centrally controls an operation of each part of the imaging console 2 and the radiation irradiation operation and the reading operation by the imaging device 1.

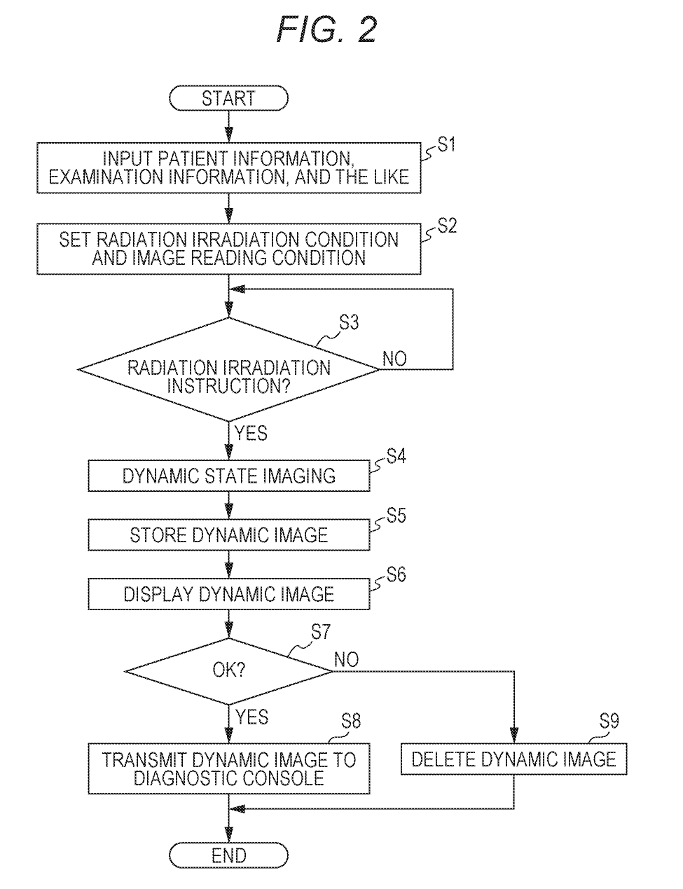

The storage 22 includes a nonvolatile semiconductor memory, hard disk, and the like. The storage 22 stores various programs executed by the controller 21, parameters necessary for execution of the programs, data of processing results, and the like. For example, the storage 22 stores a program to execute the imaging control processing illustrated in FIG. 2. In addition, the storage 22 stores the radiation irradiation conditions and the image reading conditions in association with a subject part (here, chest). Various programs are stored in a form of readable program code, and the controller 21 sequentially performs operations according to the program code.

The operator 23 includes a keyboard including a cursor key, numeric input keys, various function keys, and the like and a pointing device such as a mouse, and outputs an instruction signal input by a key operation on the keyboard and a mouse operation to the controller 21. Furthermore, the operator 23 may include a touch panel on a display screen of the display 24. In this case, the operator 23 outputs an instruction signal input via the touch panel to the controller 21.

The display 24 includes a monitor such as a liquid crystal display (LCD) and a cathode ray tube (CRT) and displays the input instruction from the operator 23, data, and the like according to an instruction of a display signal input from the controller 21.

The communicator 25 includes a LAN adapter, a modem, a terminal adapter (TA), and the like and controls data transmission/reception with each device connected to the communication network NT.

### [Configuration of Diagnostic Console 3]

The diagnostic console 3 is a dynamic image processing device which obtains the dynamic image from the imaging console 2 and assists diagnosis by a doctor by displaying the obtained dynamic image and the analysis results of the dynamic image.

As illustrated in FIG. 1, the diagnostic console 3 includes a controller 31, a storage 32, an operator 33, a display 34, and a communicator 35, and these parts are connected with a bus 36.

The controller 31 includes a CPU, a RAM, and the like. The CPU of the controller 31 reads a system program and various programs stored in the storage 32 and develops the read program in the RAM in response to an operation of the operator 33, performs various processing including comparison display processing A to be described later according to the developed program, and centrally controls an operation of each part of the diagnostic console 3. The controller 31 functions as a setter, a frame image extractor, dynamic state dependent characteristic amount extractor, and a standardizer.

The storage 32 includes a nonvolatile semiconductor memory, a hard disk, and the like. The storage 32 stores various programs including a program necessary for executing the comparison display processing A by the controller 31, parameters necessary for execution of the programs, data of processing results, or the like. The various programs are stored in a form of readable program code, and the controller 31 sequentially performs operations according to the program code.

In addition, the storage 32 stores a dynamic image imaged in the past in association with patient information (for example, patient ID, patient's name, height, weight, age, and gender), examination information (for example, examination ID, examination date, subject part (here, chest), and kind of dynamic state to be diagnosed (for example, quiet breathing, deep breathing, and heartbeat)). Alternatively, electronic medical chart information corresponding to the dynamic image may be obtained from an electronic medical chart device which is not illustrated and stored in association with the dynamic image.

The operator 33 includes a keyboard including a cursor key, numeric input keys, various function keys, and the like and a pointing device such as a mouse, and outputs an instruction signal input by a key operation on the keyboard and a mouse operation by a user to the controller 31. Furthermore, the operator 33 may include a touch panel on a display screen of the display 34. In this case, the operator 33 outputs an instruction signal input via the touch panel to the controller 31.

The display 34 includes a monitor such as an LCD and a CRT, and performs various displays according to an instruction of a display signal input from the controller 31.

The communicator 35 includes a LAN adapter, a modem, a TA, and the like and controls data transmission/reception with each device connected to the communication network NT.

### [Operation of Dynamic Image Processing System 100]

Next, an operation of the dynamic image processing system 100 according to the present embodiment will be described.

### (Operations of Imaging Device 1 and Imaging Console 2)

First, imaging operations of the imaging device 1 and the imaging console 2 will be described.

FIG. 2 illustrates imaging control processing executed by the controller 21 of the imaging console 2. The imaging control processing is executed by the controller 21 in cooperation with programs stored in the storage 22,

First, a photographer operates the operator 23 of the imaging console 2 and inputs the patient information and the examination information of an examinee (step S1).

Next, the radiation irradiation condition is read from the storage 22 and is set to the radiation irradiation control device 12, and the image reading condition is read from the storage 22 and is set to the reading control device 14 (step S2).

Then, a radiation irradiation instruction by an operation of the operator 23 is waited (step S3). Here, the photographer arranges the subject M between the radiation source 11 and the radiation detector 13 and performs positioning. Furthermore, the photographer instructs the examinee to be in a breathing state according to the kind of the dynamic state to be diagnosed. At the time when preparation for imaging is completed, the radiation irradiation instruction is input by operating the operator 23.

When the radiation irradiation instruction is input by the operator 23 (step S3; YES), an imaging start instruction is output to the radiation irradiation control device 12 and the reading control device 14, and the dynamic state imaging is started (step S4). That is, radiation is emitted by the radiation source 11 at the pulse intervals set by the radiation irradiation control device 12, and the radiation detector 13 obtains a frame image.

When the predetermined number of frames is imaged, the controller 21 outputs an instruction to end the imaging to the radiation irradiation control device 12 and the reading control device 14, and the imaging operation is stopped. The number of frames to be imaged is the number of images that can image at least a single breathing cycle.

The frame images obtained by imaging are sequentially input to the imaging console 2, and stored in the storage 22 in association with numbers (frame number) indicating imaging order (step S5), and displayed on the display 24 (step S6). The photographer confirms positioning and the like according to the displayed dynamic image and determines whether the image suitable for diagnosis is imaged (imaging OK) or it is necessary to image the dynamic image again (imaging not OK). Then, the determination result is input by operating the operator 23.

When the determination result indicating that imaging is OK is input by a predetermined operation of the operator 23 (step S7; YES), information such as an identification ID for identifying the dynamic image, the patient information, the examination information, the radiation irradiation conditions, the image reading conditions, the number indicating the imaging order (frame number), and the like is attached to each of the series of frame images obtained by dynamic state imaging (for example, information in DICOM format is written to header region of image data) and is transmitted to the diagnostic console 3 via the communicator 25 (step S8). Then, this processing is terminated. On the other hand, when the determination result indicating that imaging is not OK is input by a predetermined operation of the operator 23 (step S7; NO), the series of frame images stored in the storage 22 is deleted (step S9), and this processing is terminated. In this case, re-imaging is required.

### (Operation of Diagnostic Console 3)

Next, an operation of the diagnostic console 3 will be described.

When the diagnostic console 3 receives the series of frame images of the dynamic image from the imaging console 2 via the communicator 35, the received dynamic image is stored in the storage 32.

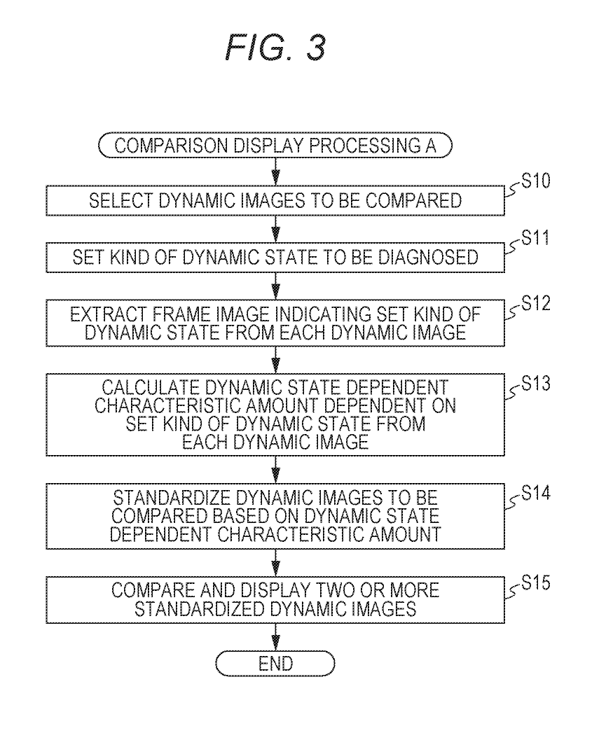

When the operator 33 instructs to start comparative diagnostic reading, the comparison display processing A illustrated in FIG. 3 is executed by the controller 31 in cooperation with the programs stored in the storage 32.

Hereinafter, a flow of the comparison display processing A will be described with reference to FIG. 3.

First, two or more dynamic images to be compared are selected (step S10).

In step S10, for example, a list of dynamic images of the subject M stored in the storage 32 is displayed on the display 34, and the operator 33 selects a dynamic image desired by a user from among the displayed dynamic images.

Next, the kind of the dynamic state to be diagnosed is set by the user's operation (step S11).

In step S11, for example, a setting screen is displayed on the display 34, and the kind of the dynamic state to be diagnosed is set according to the operation of the operator 33 by the user on the setting screen. For example, in a case where a subject part is the chest, options such as quiet breathing, deep breathing, and heartbeat (breathing hold) are displayed on the setting screen, and the kind of the dynamic state selected by the operator 33 is set as the kind of the dynamic state to be diagnosed. Alternatively, the user may optionally input the kind of the dynamic state.

In the following description, an example will be described in which any one of quiet breathing, deep breathing, and heartbeat (breathing hold) can be set as the kind of the dynamic state to be diagnosed.

Next, a frame image indicating the dynamic state of the kind set in step S11 is extracted from the dynamic images to be compared (step S12).

For example, a predetermined characteristic amount is extracted from each dynamic image, and the frame image in the section indicating the dynamic state of the kind set in step S11 is extracted from the series of frame images of each dynamic image based on the predetermined characteristic amount.

In step S11, for example, a waveform indicating a temporal change in a position of a structure (position of characteristic point of structure) which moves in association with respiration (lung) such as the diaphragm, ribs, outer thorax, and soft tissue (breast and the like) is extracted as a characteristic amount from each frame image of the dynamic image. In a case Where the set kind of the dynamic state is quiet breathing, a frame image, of which a period of the extracted waveform is constant, in a section shorter than a predetermined threshold is extracted. In a case where the set kind of the dynamic state is deep breathing, a frame image, of which a period of the extracted waveform is constant, in a section longer than the predetermined threshold is extracted. In a case where the set kind of the dynamic state is heartbeat, a frame image in a section in which the extracted waveform is not changed (breathing hold section, section in which dynamic state of heartbeat appears with no effect of breathing) is extracted.

A region of the structure such as the diaphragm, the ribs, the outer thorax, the soft tissue (breast and the like), and the like can be detected by using a known image processing technique such as an edge detection technique.

Furthermore, a contrast agent improves performance for visualizing blood vessels, and a pulmonary blood vessel is a structure which moves along with the lung. Therefore, a region having a predetermined concentration value indicating the contrast agent is detected from each frame image of the dynamic image, and a waveform indicating a temporal change in a position of a predetermined characteristic point in the detected region may be used as a characteristic amount instead of the waveform indicating the temporal change in the position of the structure.

Furthermore, a waveform indicating a temporal change in a concentration value of a lung field region based on ventilation (representative value of concentration value of each pixel in lung field region) is generated, and the generated waveform may be used as a characteristic amount instead of the waveform indicating the temporal change in the position of the structure. The concentration value of the lung field region based on the ventilation can be obtained by performing low-pass filtering in a time direction (for example, cutoff frequency of 0.8 Hz) on the waveform indicating the temporal change in the concentration value of the lung field region.

Furthermore, a waveform indicating a temporal change in an inter-frame difference value between a concentration value of the lung field region based on ventilation in each frame image and a concentration value of a lung field region based on the ventilation in a reference frame image (for example, frame image with maximum expiration value or maximum inspiration value) may be used as a characteristic amount instead of the waveform indicating the temporal change in the position of the structure.

Then, a dynamic state dependent characteristic amount dependent on the dynamic state of the kind set to be diagnosed in step S11 is calculated from each dynamic image (dynamic image including extracted frame image) (step S13).

For example, in a case where quiet breathing or deep breathing is set in step S11, as the dynamic state dependent characteristic amount dependent on the set kind of the dynamic state, for example, the waveform indicating the temporal change in the position of the structure such as the ribs, the outer thorax, the diaphragm, or the soft tissue which moves along with quiet breathing and deep breathing, the waveform indicating the temporal change in the position of the pulmonary blood vessel visualized by the contrast agent, and the waveform indicating the temporal change in the concentration value of the lung field region based on ventilation and the difference value between the concentration values of the respective frames are calculated. Since a method of extracting these characteristic amounts is similar to those described in step S12, the description thereof will be cited.

In a case where the heartbeat is set in step S11, as the dynamic state dependent characteristic amount dependent on the set kind of the dynamic state, the characteristic amount indicating the dynamic state which changes along with the heartbeat, for example, a temporal change in an area of a heart region, a temporal change in a concentration value of the heart area, a temporal change in expansion and contraction of a pulmonary blood vessel region, a temporal change in a concentration value of the pulmonary blood vessel region is calculated.

The area of the heart region can be obtained, for example, by extracting the heart region from the frame image and counting the number of pixels in the extracted region. The heart region can be detected by using a known image processing technique such as template matching processing using a template image of the heart and the like.

The temporal change in the expansion and contraction of the pulmonary blood vessel can be obtained, for example, by obtaining a temporal change in a thickness of the pulmonary blood vessel by obtaining a difference between the frame images. Alternatively, it is possible to extract the pulmonary blood vessel region to obtain the temporal change in the thickness of the pulmonary blood vessel. The pulmonary blood vessel region can be detected, for example, by using a known image processing technique using a filter for extracting a pulmonary blood vessel model and a line structure. In addition, as described above, the pulmonary blood vessel region may be detected by detecting a region having a concentration value indicating the contrast agent.

Then, based on the dynamic state dependent characteristic amount dependent on the set kind of the dynamic state to be diagnosed, which has been calculated in step S13, dynamic images to be compared are standardized (step S14).

For example, based on a period and a phase of the dynamic state dependent characteristic amount calculated from each dynamic image in step S13, a period and a phase of the dynamic state to be diagnosed in each dynamic image are standardized to meet a predetermined standard. For example, the period and the phase of the dynamic state dependent characteristic amount calculated from each dynamic image meet the predetermined standard so that the periods and the phases of the dynamic states to be diagnosed in the respective dynamic images can be the same.

The standards of the period and the phase may be the period and the phase of any one of the dynamic images, and a predetermined standard may be provided.

For example, in a case where a standard of a dynamic image of which the period of the dynamic state dependent characteristic amount is short is met a standard of a dynamic state dependent characteristic amount having a long period, the period of the dynamic state dependent characteristic amount is met the standard period by up-sampling the dynamic image in the time direction (add frame images evenly in time direction) so that the period of the dynamic state dependent characteristic amount of each dynamic image matches the standard period. A pixel value (concentration value) of each pixel of the frame image to be added can be obtained, for example, by interpolation processing by using pixel values of pixels at the same position in the multiple frame images of the original dynamic image. In addition, in a case where a standard of a dynamic image of which a period of the dynamic state dependent characteristic amount is long is met a standard of a dynamic state dependent characteristic amount having a short period, the period of the dynamic state dependent characteristic amount is met the standard period by down-sampling the dynamic image in the time direction (thin out (delete) frame images evenly in time direction).

After the periods have been met, based on the dynamic state dependent characteristic amount of each dynamic image, a shift amount in the time direction of the dynamic image to make the phase of the dynamic state to be diagnosed at a start timing of each dynamic image coincide with a predetermined standard phase (for example, maximum point or minimum point) is calculated, and the frame image of each dynamic image is shifted in the time direction by the calculated shift amount. Accordingly, it is possible to match the phases of the dynamic states to be diagnosed at the start timings of the respective dynamic images to be compared.

Then, two or more standardized dynamic images are compared and displayed on the display 34 (step S15), and the comparison display processing A is terminated.

In step S15, example, the two or more standardized dynamic images are displayed side by side on the display 34.

For example, two or more dynamic images are displayed in order of imaged dates (examination date). Alternatively, the dynamic images may be displayed in order based on a predetermined item in the electronic medical chart information such as a dosage, a disease stage, a height, a weight, and the like. Alternatively, the dynamic images may be displayed in order based on the dynamic state dependent characteristic amount calculated in step S13 (for example, order based on moving speed of structure).

Alternatively, the two or more standardized dynamic images having different colors may be superimposed and displayed.

Furthermore, in step S15, a difference between the corresponding frame images of the two or more standardized dynamic images is calculated, and a difference image may be generated and displayed. The difference may be emphasized by coloring the difference and the like, Furthermore, the frame images of the difference image (or dynamic image) may be displayed in descending order of the difference, and only the frame images of the difference image (or dynamic image) having a large difference (larger than predetermined threshold) may be displayed in slow motion or may be extracted and displayed. Alternatively, a frame image having a large difference may be formed as a thumbnail and displayed as a representative image of each dynamic image. Since this facilitates easy recognition of the difference between the dynamic states to be diagnosed in the respective dynamic images to be compared, a doctor can easily perform comparative diagnostic reading.

As described above, in the first embodiment, since the two or more dynamic images to be compared are standardized based on the dynamic state dependent characteristic amount dependent on the set kind of the dynamic state, the periods and the phases of the set kind of the dynamic state can be aligned and displayed, and the doctor can easily perform comparative diagnostic reading on the dynamic state to be diagnosed.

### Second Embodiment

Hereinafter, a second embodiment of the present invention will be described.

A configuration of the second embodiment is similar to that described in the first embodiment except for that a program for executing comparison display processing B is stored in a storage 32 of a diagnostic console 3. Therefore, description thereof will be omitted, and an operation of the second embodiment will be described below.

First, dynamic state imaging is performed by an imaging device 1 and an imaging console 2 to generate a dynamic image, and the imaging console 2 transmits a series of frame images of the dynamic image to the diagnostic console 3.

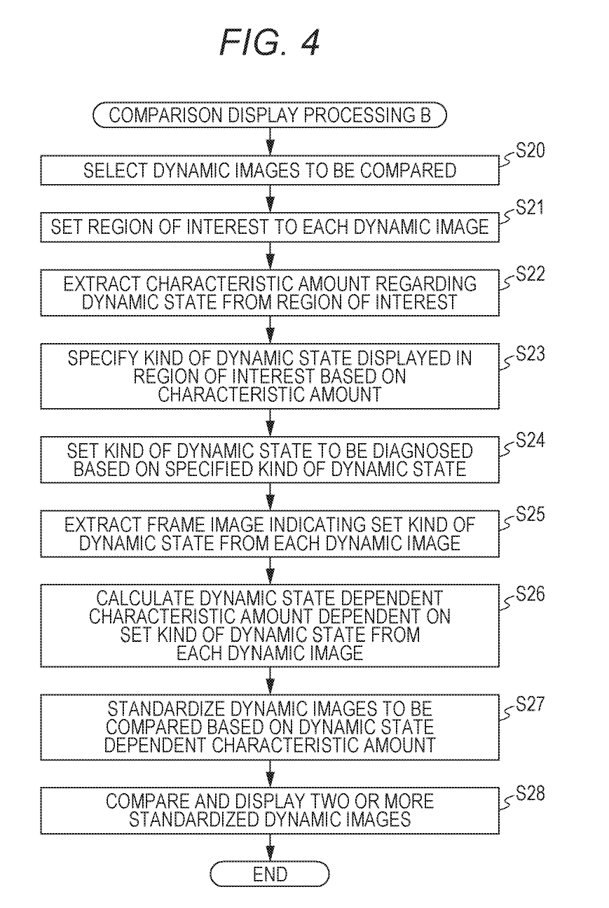

When the diagnostic console 3 receives the series of frame images of the dynamic image from the imaging console 2 via a communicator 35, the received series of frame images is stored in the storage 32. Furthermore, when the operator 33 instructs to start comparative diagnostic reading, the comparison display processing B illustrated in FIG. 4 is executed by a controller 31 in cooperation with the programs stored in the storage 32.

FIG. 4 is a flowchart illustrating the comparison display processing B executed by the diagnostic console 3 in the second embodiment. The comparison display processing B is executed by the controller 31 in cooperation with the programs stored in the storage 32.

First, two or more dynamic images to be compared are selected (step S20).

Since the processing in step S20 is similar to the processing described in step S10 in FIG. 3, the description is cited.

Next, a region of interest is set to each dynamic image (step S21).

For example, the entire image may be set as a region of interest, and a region designated by a user in the dynamic image via the operator 33 may be set as a region of interest. Furthermore, a predetermined structure (for example, lung field region, heart region, and the like) may be automatically detected from a dynamic image to set the detected region as a region of interest.

Next, a characteristic amount regarding the dynamic state is extracted from the region of interest (step S22).

For example, the characteristic amount regarding the dynamic state is extracted from the region of interest of each dynamic image to be compared. For example, in a case where the region of interest is a lung field region, a waveform indicating a temporal change in a concentration value of the lung field region based on ventilation (representative value of each pixel in lung field region) or a difference value different from a reference frame image and the like is extracted as a characteristic amount.

Next, a kind of a dynamic state displayed in the region of interest is specified based on the characteristic amount extracted from the region of interest of each dynamic image (step S23).

For example, in a case where the region of interest is the lung field and a period of the characteristic amount (waveform) extracted in step S22 in each dynamic image is constant and shorter than a predetermined threshold, the kind of the dynamic state displayed in the region of interest is specified as quiet breathing. In a case where the period of the characteristic amount (waveform) is constant and longer than the predetermined threshold, the kind of the dynamic state displayed in the region of interest is specified as deep breathing. In a case where the waveform does not change, the kind of the dynamic state displayed in the region of interest is specified as breathing hold.

In a case where a dynamic image exists which has a section with a plurality of characteristics from among (1) the period of the characteristic amount is constant and shorter than the threshold, (2) the period of the characteristic amount is constant and longer than the threshold, and (3) the waveform does not change, the kind of the dynamic state corresponding to the characteristics common to the dynamic images to be compared is specified as the kind of the dynamic state displayed in the region of interest. In a case where the plurality of characteristics common to the dynamic images to be compared exists, the kind of the dynamic state displayed in the region of interest is specified according to a predetermined rule, for example, to specify the kind of the dynamic state corresponding to the longest section as the kind of the dynamic state displayed in the region of interest.

Next, a kind of the dynamic state to be diagnosed is set based on the specified kind of the dynamic state (step S24).

For example, in a case where the specified kind of the dynamic state is quiet breathing, the kind of the dynamic state to be diagnosed is set to quiet breathing. In a case where the specified kind of the dynamic state is deep breathing, the kind of the dynamic state to be diagnosed is set to deep breathing. In a case where the specified kind of the dynamic state is breathing hold, the kind of the dynamic state to be diagnosed is set to heartbeat.

When the kind of the dynamic state to be diagnosed is set, processing in steps S25 to S28 is executed. Since the processing in steps S25 to S28 is similar to the processing in steps S12 to S15 in FIG. 3 described in the first embodiment, the description is cited.

In this way, in the second embodiment, the kind of the dynamic state to be diagnosed can be automatically set according to the dynamic state in the region of interest.

### Third Embodiment

Hereinafter, a third embodiment of the present invention will be described.

A configuration of the third embodiment is similar to that described in the first embodiment except for that a program for executing comparison display processing C is stored in a storage 32 of a diagnostic console 3. Therefore, description thereof will be omitted, and an operation of the third embodiment will be described below.

First, dynamic state imaging is performed by an imaging device 1 and an imaging console 2 to generate a dynamic image, and the imaging console 2 transmits a series of frame images of the dynamic image to the diagnostic console 3.

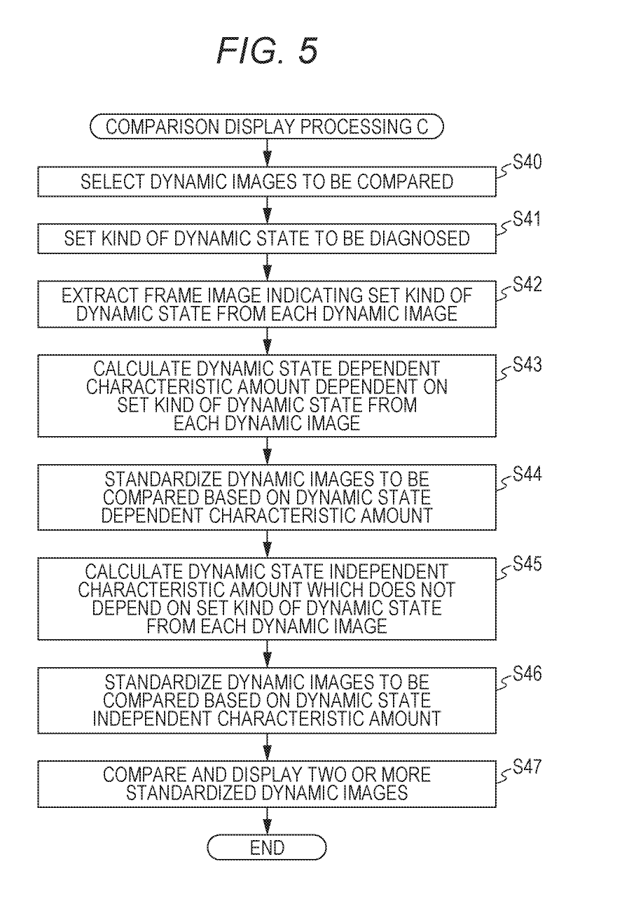

When the diagnostic console 3 receives the series of frame images of the dynamic image from the imaging console 2 via a communicator 35, the received series of frame images is stored in the storage 32. Furthermore, when the operator 33 instructs to start comparative diagnostic reading, the comparison display processing C illustrated in FIG. 5 is executed by a controller 31 in cooperation with the programs stored in the storage 32.

FIG. 5 is a flowchart illustrating the comparison display processing C executed by the diagnostic console 3 in the third embodiment. The comparison display processing C is executed by the controller 31 in cooperation with the programs stored in the storage 32.

First, two or more dynamic images to be compared are selected (step S40).

Next, a kind of a dynamic state to be diagnosed is set by the user's operation (step S41).

Next, a frame image in a section indicating the dynamic state of the kind set in step S41 is extracted from the dynamic images to be compared (step S42).

Then, a dynamic state dependent characteristic amount dependent on the dynamic state of the kind set to be diagnosed in step S41 is calculated from each dynamic image (dynamic image including extracted frame image)(step S43).

Then, based on the dynamic state dependent characteristic amount dependent on the kind of the dynamic state set to be diagnosed, which has been calculated in step S43, dynamic images to be compared are standardized (step S44).

Since the details of steps S40 to S44 are the same as those described in steps S10 to S14 in FIG. 3, the description is cited.

Subsequently, a dynamic state independent characteristic amount which does not depend on the set kind of the dynamic state is calculated from each dynamic image to be compared (dynamic image including extracted frame image)(step S45).

The dynamic state independent characteristic amount which does not depend on the set kind of the dynamic state is a characteristic amount which does not change according to the set kind of the dynamic state and is, for example, a characteristic amount regarding the position of the predetermined structure and/or the concentration value of the dynamic image. For example, a position of the backbone, a central position of the heart, a contour position of a body surface, an average concentration and dispersion of the entire dynamic image of the structure to be diagnosed are exemplified.

The position of the backbone can be obtained by extracting an edge along a vertical direction in the vicinity of the center in the horizontal direction of the image, for example, by using a known edge extraction method. The contour of the body surface can be obtained by binarizing each frame image and extracting a boundary extending along the vertical direction near the side end of the image.

Next, the dynamic images to be compared are further standardized based on the calculated dynamic state independent characteristic amount (step S46).

In step S46, an element (position of predetermined structure and/or concentration value and the like) which does not change according to the set kind of the dynamic state of the dynamic images to be compared is standardized. For example, based on the dynamic state independent characteristic amount, affine transformation is performed so that the characteristic amounts such as the position of the backbone, the central position of the heart, or the contour position of the body surface of the corresponding frame images of the dynamic image coincide with each other (to meet predetermined standard), the subject is moved, rotated, and expanded and contracted.

In addition, a contrast is adjusted so that the average concentrations and the dispersions of the entire dynamic images of the structure region regarding the dynamic state to be diagnosed (for example, lung field region in a case where kind of dynamic state to be diagnosed is deep breathing or quiet breathing and heart region in a case where kind of dynamic state to be diagnosed is heartbeat) coincide with each other meet predetermined standard).

Then, two or more standardized dynamic images are compared and displayed on the display 34 (step S47), and the comparison display processing C is terminated.

Since details of step S47 is similar to that described in step S15 in FIG. 3, the description is cited.

As described above, in the third embodiment, since the two or more dynamic images to be compared are standardized based on the dynamic state dependent characteristic amount dependent on the set kind of the dynamic state, the periods and the phases of the set kind of the dynamic state can be aligned and displayed, and a doctor can easily perform comparative diagnostic reading on the dynamic state to be diagnosed. Furthermore, since the two or more dynamic images to be compared are standardized based on the dynamic state independent characteristic amount which does not change according to the set kind of the dynamic state, elements of the two or more dynamic images to be compared which do not change according to the set kind of the dynamic state (position of structure or concentration of entire dynamic image which does not change according to set kind of dynamic state) can be aligned and displayed, and a doctor can more easily perform comparative diagnostic reading.

### Fourth Embodiment

Hereinafter, a fourth embodiment of the present invention will be described.

A configuration of the fourth embodiment is similar to that described in the first embodiment except for that a program for executing comparison display processing D is stored in a storage 32 of a diagnostic console 3. Therefore, description thereof will be omitted, and an operation of the fourth embodiment will be described below.

First, dynamic state imaging is performed by an imaging device 1 and an imaging console 2 to generate a dynamic image, and the imaging console 2 transmits a series of frame images of the dynamic image to the diagnostic console 3.

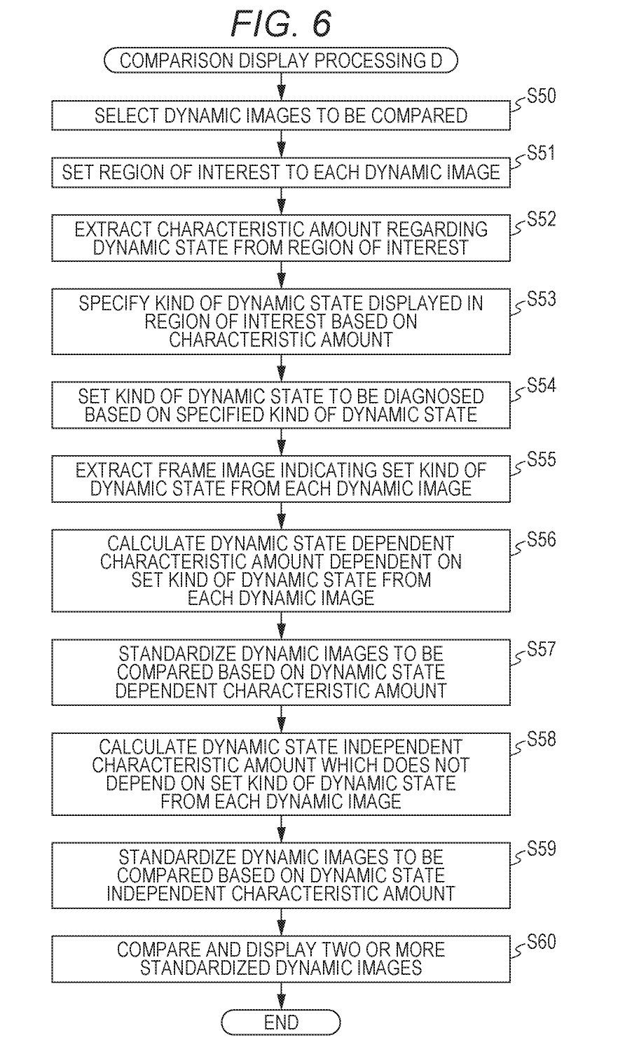

When the diagnostic console 3 receives the series of frame images of the dynamic image from the imaging console 2 via a communicator 35, the received series of frame images is stored in the storage 32. Furthermore, when an operator 33 instructs to start comparative diagnostic reading, the comparison display processing D illustrated in FIG. 6 is executed by a controller 31 in cooperation with the programs stored in the storage 32.

FIG. 6 is a flowchart illustrating the comparison display processing D executed by the diagnostic console 3 in the fourth embodiment. The comparison display processing D is executed by the controller 31 in cooperation with the programs stored in the storage 32.

First, two or more dynamic images to be compared are selected (step S50)

Next, a region of interest is set to each dynamic image (step S51).

Next, a characteristic amount regarding the dynamic state is extracted from the region of interest (step S52).

Then, the kind of the dynamic state displayed in the region of interest is specified based on the characteristic amount extracted from each dynamic image (step S53), and the kind of the dynamic state to he diagnosed is set based on the specified kind of the dynamic state (step S54).

Since the processing in steps S50 to S54 is similar to the processing in steps S20 to S24 described in the second embodiment, the description is cited.

When the kind of the dynamic state to be diagnosed is set, processing in steps S55 to S60 is executed. Since the processing in steps S55 to S60 is similar to the processing in steps S42 to S47 in FIG. 5 described in the third embodiment, the description is cited. That is, the dynamic images to be compared are standardized based on the dynamic state dependent characteristic amount of the set kind of the dynamic state, and the dynamic images to be compared are standardized based on the dynamic state independent characteristic amount, and the standardized dynamic images are compared and displayed on the display 34.

In this way, in the fourth embodiment, the kind of the dynamic state to be diagnosed can be automatically set according to the dynamic state in the region of interest. Furthermore, since the two or more dynamic images to be compared are standardized based on the dynamic state dependent characteristic amount dependent on the set kind of the dynamic state, the periods and the phases of the set kind of the dynamic state can be aligned and displayed, and a doctor can easily perform comparative diagnostic reading on the dynamic state to be diagnosed. Furthermore, since the two or more dynamic images to be compared are standardized based on the dynamic state independent characteristic amount which does not change according to the set kind of the dynamic state, elements of the two or more dynamic images to be compared which do not change according to the set kind of the dynamic state (position of structure or concentration of entire dynamic image which does not change according to set kind of dynamic state) can be aligned and displayed, and a doctor can more easily perform comparative diagnostic reading.

Although embodiments of the present invention have been described and illustrated in detail, the disclosed embodiments are made for purposes of illustration and example only and not limitation. The scope of the present invention should be interpreted by terms of the appended claims.

For example, in the embodiments, an example has been described in which the subject part is the chest. However, in a case where a plurality of dynamic images obtained by imaging other parts is compared, the present invention can be applied.

Furthermore, the kind of the dynamic state which can be set as a diagnosis target and various characteristic amounts are not limited to the examples in the embodiments.

In the third and fourth embodiments described above, it has been described that the dynamic state independent characteristic amount is calculated and the dynamic images are standardized based on the dynamic state independent characteristic amount after calculating the dynamic state dependent characteristic amount and standardizing the dynamic images based on the dynamic state dependent characteristic amount. However, it is possible that the dynamic state dependent characteristic amount is calculated and the dynamic images are standardized based on the dynamic state dependent characteristic amount after calculating the dynamic state independent characteristic amount and standardizing the dynamic images based on the dynamic state independent characteristic amount. Furthermore, standardization based on each characteristic amount may be performed after the dynamic state dependent characteristic amount and the dynamic state independent characteristic amount have been calculated.

Furthermore, for example, in the above description, an example has been disclosed in which a hard disk, a nonvolatile memory of a semiconductor, and the like are used as a computer readable medium of the program according to the present invention. However, the present invention is not limited to this example. As the other computer recording medium, a portable readable medium such as a CD-ROM can be applied. Carrier wave (carrier) is applied as a medium for providing data of the program according to the present invention via a communication line.

In addition, the detailed structure and the detailed operation of each device included in the dynamic image processing system can be appropriately changed without departing from the spirit of the present invention.

What is claimed is:

1. A dynamic image processing device comprising:
a hardware processor that
sets a kind of a dynamic state to be diagnosed;
extracts a frame image indicating the dynamic state of the kind set by the hardware processor from each of a plurality of dynamic images obtained by imaging a dynamic state of a living body with radiation;
extracts a dynamic state dependent characteristic amount dependent on the dynamic state of the kind set by the hardware processor from the frame image extracted from each of the plurality of dynamic images; and
standardizes the plurality of dynamic images based on the dynamic state dependent characteristic amount extracted by the hardware processor.

2. The dynamic image processing device according to claim 1, wherein
the hardware processor sets the kind of the dynamic state to be diagnosed according to a user's operation.

3. The dynamic image processing device according to claim 1, wherein
the hardware processor
sets a region of interest in each of the plurality of dynamic images;
extracts a characteristic amount regarding a dynamic state from the region of interest set by the hardware processor; and
specifies a kind of the dynamic state displayed in the region of interest based on the characteristic amount extracted by the hardware processor, and
the hardware processor sets the kind of the dynamic state to be diagnosed based on the kind of the dynamic state specified by the hardware processor.

4. The dynamic image processing device according to claim 1, wherein
the hardware processor performs standardization for aligning periods and phases of the dynamic states to be diagnosed of the plurality of dynamic images based on the dynamic state dependent characteristic amount.

5. The dynamic image processing device according to claim 1, wherein
the hardware processor extracts a dynamic state independent characteristic amount Which does not depend on the kind oldie dynamic state set by the hardware processor from each of the plurality of dynamic images, and
the hardware processor further standardizes the plurality of dynamic images based on the dynamic state independent characteristic amount.

6. The dynamic image processing device according to claim 5, wherein
the dynamic state independent characteristic amount is a characteristic amount regarding a position of a predetermined structure and/or a concentration value of the dynamic image, and
the hardware processor standardizes positions of the predetermined structures and/or concentration values of the plurality of dynamic images based on the dynamic state independent characteristic amount.

7. The dynamic image processing device according to claim 1, wherein
the hardware processor displays the plurality of dynamic images standardized by the hardware processor side by side or to be overlapped with each other.

8. The dynamic image processing device according to claim 1, wherein
the hardware processor displays a difference image of the plurality of dynamic images standardized by the hardware processor.
